(19) United States
(12) Plant Patent Application Publication
(10) Pub. No.: US 2003/0028944 P1
(43) Pub. Date: Feb. 6, 2003

(54) Gladiolus plant named 'Euro Blanca'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/918,663

(22) Filed: Jul. 31, 2001

(51) Int. Cl. A01H 5/00

(52) U.S. Cl. PLT/301

(57) ABSTRACT

A new and distinct gladiolus variety, designated ‘Euro Blanca’, is shown and described. Compared to the ‘T-200 variety, the ‘Euro Blanca’ variety has a stem length that is about 15-20 cm longer, contains 1 to 2 more florets per flower head, and can hold open five to six blooms similar to the ‘T-200’ variety.

The present invention comprises a new and distinct variety of a Gladiolus plant referred to by the variety name ‘Euro Blanca.’

### BRIEF DESCRIPTION OF THE PHOTOGRAPHS

FIG. 1 is a photograph of a ‘Euro Blanca’ variety plant in bloom.

### ASEXUAL REPRODUCTION

The new variety was originated by the Applicant in a controlled proprietary breeding program in Ft. Myers, Fla. wherein selected gladiolus varieties were crossed (the Applicant's selected provisional breeder's designation is 38-1). The female parent was a white gladiolus variety named ‘T-200,’ an unpatented, released variety by Turk characterized in part by having a harvesting time similar to the ‘White Friendship’ gladiolus variety, 16 to 18 florets, and lightweight stems. The male parent was a green gladiolus variety named ‘T-815,’ released variety characterized in part by having a long head, small blooms, and poor opening ability. The seeds were planted in Ft. Myers, Fla., and the selection of the ‘Euro Blanca’ variety was made in Spring 1988. Asexual reproduction of the ‘Euro Blanca’ variety was achieved by collecting cormels from the first corm. All subsequent asexual reproductions of the ‘Euro Blanca’ variety remain true to the original variety type. Botanical Description:

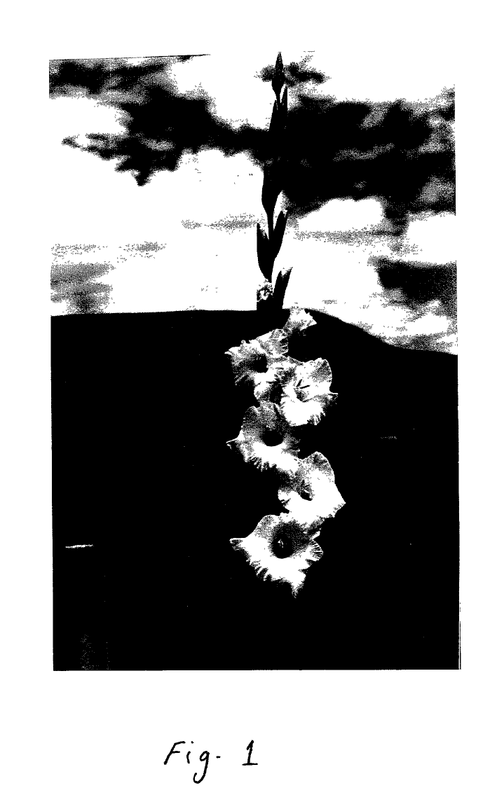

The accompanying color photograph (FIG. 1) shows the inflorescence and various stages of blooming of the ‘Euro Blanca’ variety plant.

The bud size of the ‘Euro Blanca’ variety is about 6.5 cm in length. The flowers of the variety each consist of a total of six petals, namely five large petals and one small petal located on the lower half of the flower (i.e. at about 6:00). The variety has a light green color deep in the throat, followed by a uniform white color throughout the rest of the petals.

The spike of the plant is about 147 cm and contains 18-19 florets when grown from #2 size corms (i.e. 1.25 to 1.50 inches) in Ecuador. The head length is about 50 cm, and the bloom diameter is 10 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white, and the stamens are purple.

The corms of the ‘Euro Blanca’ variety are typical for the gladiolus.

The leaf color of the ‘Euro Blanca’ variety is green. The leaf size is about 90 cm (length) by 5 cm (width).

### GENERAL OBSERVATION:

The ‘Euro Blanca’ variety takes about 83 days to harvest in Ecuador, which is about seven days longer than the ‘T-200’ gladiolus variety. Unlike the ‘T-200’ variety, the ‘Euro Blanco’ variety provides excellent growth in mid-winter days of 10.5 hours and has good tolerance to high temperatures without burning.

The ‘Euro Blanca’ variety has normal resistance to Curvalaria and Botrytis foliar diseases, but is susceptible to attack by Fusarium species.

The variety usually produces one to two stems per #2 size corm. The variety can hold open 5-6 blooms after shipping one week in a box. The ‘Euro Blanco’ variety is 15-20 cm taller than the ‘T-200’ variety, has one to two more florets per flower head, and has a larger stem diameter than the ‘T-200’ variety. The bloom diameter of 10 cm is similar to that of the ‘T-200’ variety.

I claim:

1. A new and distinct gladiolus variety ‘euro blanca’, as shown and described herein.
